(19) United States
(12) Patent Application Publication
(10) Pub. No.: US 2015/0250483 A1
(43) Pub. Date: Sep. 10, 2015

(54) UV-CURING RESIN INJECTION DEVICE FOR ANEURISM TREATMENT

(71) Applicants: POSTECH ACADEMY-INDUSTRY FOUNDATION, Pohang-si (KR); KYUNGPOOK NATIONAL UNIVERSITY HOSPITAL, Daegu (KR)

(72) Inventors: Joon Won KIM, Pohang-si (KR); Dong-Hun KANG, Daegu (KR); Jaechan PARK, Daegu (KR); Usung PARK, Phohang-si (KR)

(21) Appl. No.: 14/696,298

(22) Filed: Apr. 24, 2015

Related U.S. Application Data
(63) Continuation of application No. PCT/KR2013/009546, filed on Oct. 24, 2013.

(30) Foreign Application Priority Data
Oct. 24, 2012 (KR) 10-2012-0118587
Oct. 23, 2013 (KR) 10-2013-0126838

(51) Int. Cl. A61B 17/12; A61F 2/86

(52) U.S. Cl. CPC A61B 17/12195; A61B 17/12118; A61F 2/86; A61B 2017/1205; A61B 2017/12054

(57) ABSTRACT

A UV curing resin injection apparatus for treating an aneurysm, includes a catheter, a light transmission tube disposed at an inner side of the catheter to transmit UV light, and a connection tube disposed at the inner side of the catheter to be spaced from the light transmission tube and configured to transfer a UV curing resin, wherein the light transmission tube is formed independently from the connection tube.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

1. Field

The present disclosure relates to a UV curing resin injection apparatus, and more particularly, to a UV curing resin injection apparatus for treating an aneurysm.

2. Related Technology

A cerebral aneurysm of circulatory diseases generated in an aneurysm is a disease in which a weak portion of a blood vessel wall swells up in the form of a ground cherry. If the blood vessel wall which is gradually thinner as a weak portion thereof swells up more and more cannot endure a blood pressure, the blood vessel wall may be ruptured, and thus a cerebral subarachnoid hemorrhage may occurs.

The cerebral aneurysm may be treated by two methods of a surgical operation and an endovascular operation. The surgical operation is a method in which a skull is opened, and a neck of the aneurysm is clamped by a clip, and the endovascular operation is a method in which a catheter is inserted into the aneurysm, and then an embolic substance is filled therein.

According to a research result of International Subarachnoid Aneurysm Trial (ISAT) in 2002, in the case of a ruptured cerebral aneurysm, it was published that an endovascular coil treatment of the endovascular operation was a safety treatment method which could reduce a risk of a patient after the treatment, and also could increase an independent survival rate after the treatment, compared with the surgical operation.

Based on the research result, the most widely used aneurysm endovascular treatment method is a method using a frame, in which a frame fitting in the aneurysm is selected and inserted into the aneurysm through a catheter, and other smaller frames are packed at an inner side of the frame so as to block an inner side of the aneurysm from a blood flow.

However, when the neck of the aneurysm is very wide, the inserted frame may be moved to a parent blood vessel, instead of staying in the aneurysm. For addressing this, there have been proposed various auxiliary operation methods in which a balloon or a stent is used, or two frames are used at the same time.

However, when the frame is used, it is difficult to completely pack the inner side the aneurysm, and also the frame may be compressed, as time goes on, and thus the aneurysm may be relapsed. Furthermore, in a computed tomography (CT) scan or a magnetic resonance imaging (MRI) scan, the frame may cause interference and thus may bring a difficult in use of an imaging equipment. For addressing this, U.S. Pat. No. 6,576,000 discloses a device in which a UV curing resin is filled in an internal space of an aneurysm, and then UV light is radiated thereon.

### SUMMARY

One aspect of the present invention provides a UV curing resin injection apparatus for treating an aneurysm, which has the following functions:

First, it is possible to provide a cerebral aneurysm endovascular treatment tool which is capable of more effectively filling an internal space of the aneurysm.

Second, it is possible to provide a treatment tool which is capable of being applied to another aneurysm other than the cerebral aneurysm.

Third, it is possible to effectively block an inner side of the aneurysm, and thus to prevent a relapse of the aneurysm, and also it is possible to simplify a treatment process, and thus to prevent another problem which may occur additionally.

Fourth, it is possible to provide a treatment tool which is capable of not causing interference in a scanning operation using a radiological or non-radiological imaging equipment.

One aspect of the present invention provides a UV curing resin injection apparatus for treating an aneurysm, including a catheter, a light transmission tube disposed at an inner side of the catheter to transmit UV light, and a connection tube disposed at the inner side of the catheter to be spaced from the light transmission tube and configured to transfer a UV curing resin, wherein the light transmission tube is formed independently from the connection tube.

The light transmission tube may be formed of optical fiber, and one end of the light transmission tube may protrude from one end of the catheter, and a UV light generating part may be disposed at the other end of the light transmission tube.

An optical fiber insertion hole may be formed at the inner side of the catheter, and the light transmission tube disposed in the optical fiber insertion hole may be slidably disposed in the optical fiber insertion hole so that one end of the light transmission tube extends from one end of the catheter when the light transmission tube is pressed toward the catheter.

The light transmission tube may be rotatably disposed in the optical fiber insertion hole.

A nozzle may be formed at one end of the connection tube, and the other end of the connection tube may be in communication with a UV resin supply part.

The UV resin supply part may be a pump device.

A UV blocking material may be coated on an outer surface of the connection tube or the catheter.

A plurality of curing resin injection orifices may be formed at one end of the nozzle.

The curing resin injection orifices may have different diameters from each other.

A balloon may be disposed at one side of the catheter, and a liquid injection tube which is in communication with the balloon to supply a liquid into the balloon and thus to expand the balloon may be disposed between one end and the other end of the catheter.

One side of the catheter may be disposed at an inner side or an outer side of a stent in which a plurality of wave-shaped modules are formed in a net shape.

The UV curing resin injection apparatus may further include a coupling member disposed between the light transmission tube and the connection tube to connect the light transmission tube with the connection tube.

The UV curing resin injection apparatus may further include a first blocking member movably disposed on an outer circumferential surface of the connection tube and configured to block the UV light radiated on the connection tube.

The UV curing resin injection apparatus may further include a second blocking member disposed at an outer circumferential surface of one end of the connection tube and configured to block the UV light radiated on the connection tube.

A reflective surface may be formed at one end of the light transmission tube, and the reflective surface may be inclined so that the UV light is reflected in a direction perpendicular to a lengthwise direction of the connection tube.

The reflective surface may be formed by reflection coating.

A plurality of micro-protrusions may be formed at a cross section and an outer circumferential surface of one end of the connection tube.

According to the UV curing resin injection apparatus for treating the aneurysm according to embodiments of the present invention, since the embolic substance transferred to a diseased part in a liquid state can be cured at the same time as being injected, it is possible to simplify the operation process, to reduce the operation time and to enhance accuracy of the treatment.

Also, the UV curing resin injection apparatus for treating the aneurysm of the present invention can be manufactured in a small size which can pass through a small blood vessel, while including the light transmission tube and the nozzle at the same time, and thus an application range of the treatment can be enlarged widely.

In addition, the UV curing resin injection apparatus for treating the aneurysm of the present invention can be modified and applied in a proper shape according to a structure and a size of the aneurysm and a patient's condition, can control a speed for filling the inner side of the aneurysm with the embolic substance through the flow control of the UV resin supply part, and can control a curing time or a curing degree of the UV curing resin through an intensity control of the light. Therefore, the UV curing resin injection apparatus for treating the aneurysm according to embodiments of the present invention can be actively applied to various situations, and a treatment effect of the aneurysm can be increased by the above-described characteristics, and also since the operation is performed using a non-metallic material, the interference can be prevented in a scanning operation using an imaging equipment.

### BRIEF DESCRIPTION OF THE DRAWINGS

FIG. 1 is a front view of a UV curing resin injection apparatus for treating an aneurysm according to a first embodiment of the present invention.

FIG. 2 is a cross-sectional view taken along line II-II of FIG. 1.

FIG. 3 is a side cross-sectional view of a catheter illustrated in FIG. 1.

FIGS. 4A-4C are plan views of nozzles.

FIG. 5 is a front view of a UV curing resin injection apparatus for treating an aneurysm according to a second embodiment of the present invention.

FIG. 6 is a cross-sectional view taken along line VI-VI of FIG. 1.

FIG. 7 is a perspective view of a part of a UV curing resin injection apparatus for treating an aneurysm according to a third embodiment of the present invention.

FIG. 8 is a longitudinal cross-sectional view of a catheter of a UV curing resin injection apparatus for treating an aneurysm according to a fourth embodiment of the present invention.

FIG. 9 is a transverse cross-sectional view of the catheter of the UV curing resin injection apparatus for treating the aneurysm according to the fourth embodiment of the present invention.

### DETAILED DESCRIPTION OF EMBODIMENTS

Hereinafter, a UV curing resin injection apparatus for treating an aneurysm according to embodiments of the present invention will be described in detail with reference to the accompanying drawings.

Generally, in the device disclosed in U.S. Pat. No. 6,576,000, since a tube through which the UV light is transmitted and a tube through which the UV curing resin flows have an annular shape in the catheter, the UV curing resin is cured by the UV light while flowing through a passage, and thus is not completely filled in an internal space of the aneurysm. Furthermore, the catheter should have a small diameter which can be inserted into a blood vessel, and thus it is not easy that the catheter, in which the tube through which the UV light is transmitted and the tube through which the UV curing resin flows are formed therein to have the annular shape, is formed to have a fine diameter.

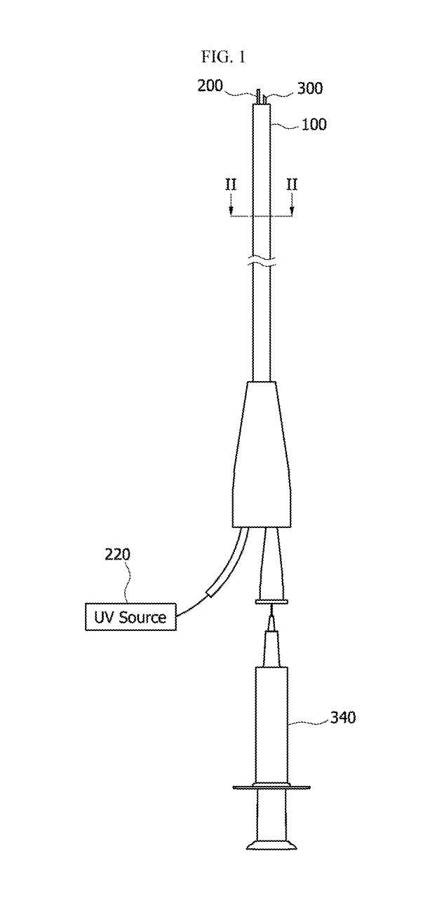

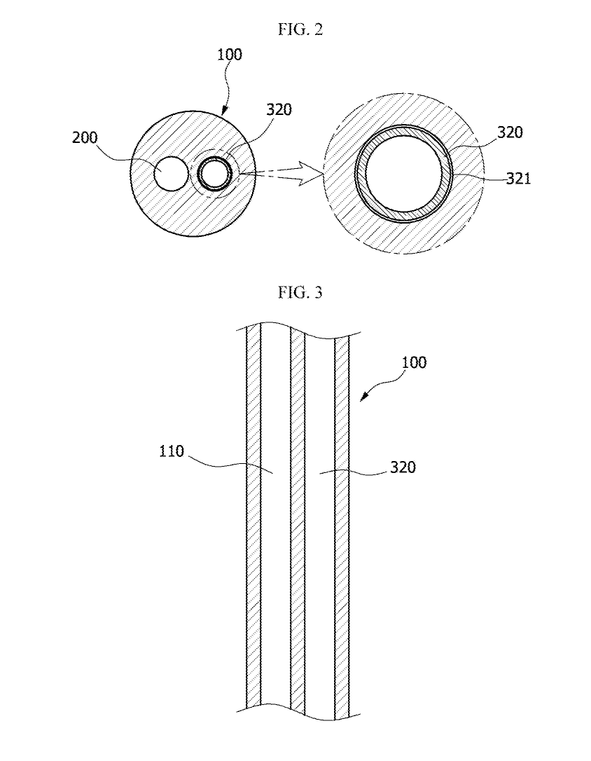

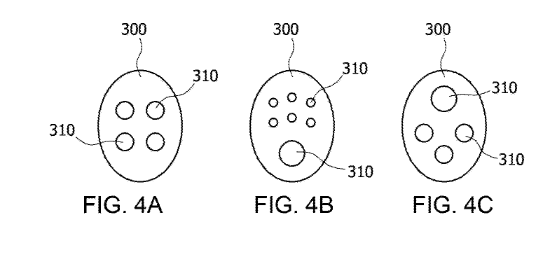

FIG. 1 is a front view of a UV curing resin injection apparatus for treating an aneurysm according to a first embodiment of the present invention, FIG. 2 is a cross-sectional view taken along line II-II of FIG. 1, FIG. 3 is a side cross-sectional view of a catheter illustrated in FIG. 1, and FIGS. 4A-4C are plan views of nozzles.

The UV curing resin injection apparatus for treating the aneurysm according to embodiments of the present invention is used in an operation for treating a cerebral aneurysm or an aneurysm, and includes a catheter 100, a light transmission tube 200 which is disposed in the catheter 100 to transmit UV light, and a connection tube or resin transfer tube 320 which is disposed in the catheter 100 to be spaced from the light transmission tube 200 and also to transfer a UV curing resin, as illustrated in FIG. 1. Also, the light transmission tube 200 is formed independently from the connection tube 320.

Therefore, when one end of the catheter 100 is inserted into the aneurysm of a blood vessel, and the UV curing resin is injected into the aneurysm through the connection tube 320, and then UV light is radiated thereon from the light transmission tube 200, the UV curing resin in the aneurysm is cured, and a blood flow introduced into the aneurysm is blocked, and thus the aneurysm is treated.

When the light transmission tube 200 according to embodiments of the present invention may be miniaturized, it may be configured with a separate UV lamp or the like. However, in the case of the embodiment, the light transmission tube 200 and a nozzle 300 may be configured with optical fiber which enables total reflection so as to be installed in the fine catheter 100 inserted into even a small vessel. One end of the light transmission tube 200 may protrude from one end of the catheter 100, and a UV light generating part or UV source 220 may be disposed at the other end thereof. Therefore, the UV light generating part 220 disposed at an outside may emit the UV light through the optical fiber to one end of the light transmission tube 200.

Referring to FIGS. 1 and 3, the above-described nozzle 300 may be in communication with a UV resin supply part 340 through the connection tube 320. When the UV curing resin is supplied, the UV resin supply part 340 may control a transferring speed and a flow rate of the UV curing resin. Therefore, the UV resin supply part 340 may be configured with a precise small pump or the like, or may be configured with a syringe which is familiar to a doctor treating the aneurysm, as illustrated in FIG. 1.

The UV curing resin used herein may be a biological adaptation UV curing resin which can be used in a human body. The UV curing resin of the embodiment was a UV curing resin in which a photoinitiator is added to polyethylene glycol diacrylate (PEGDA). Also, the UV curing resin may be mixed with a radiopaque material so as to check a position of the catheter 100 during a procedure, and may be mixed with nanoparticle clay so as to control swelling and mechanical properties of the cured UV curing resin.

Referring to FIG. 2, the connection tube 320 according to embodiments of the present invention has a circular cross section. However, the invention is not limited thereto. The connection tube 320 may have various shapes according to other embodiments.

As illustrated in FIG. 3, an optical fiber insertion hole 110 is formed in the catheter 100 according to the present invention so that the optical fiber protrudes from one end and the other end of the catheter 100. Also the light transmission tube 200 disposed in the optical fiber insertion hole 110 is slidably disposed in the optical fiber insertion hole 110 so that one end of the light transmission tube 200 extends from one end of the catheter 100 when the light transmission tube 200 is pressed toward the catheter 100.

Since a shape of the aneurysm may have various shapes, the UV curing resin injected into the aneurysm may be located to have various shapes. Therefore, one end of the light transmission tube 200 may extend from one end of the catheter 100 to effectively cure the UV curing resin located to have various shapes.

Therefore, the optical fiber insertion hole 110 may be disposed in the catheter 100 so that the light transmission tube 200 is slidably disposed therein.

Also, the light transmission tube 200 disposed in the optical fiber insertion hole 110 may be rotatably disposed in the optical fiber insertion hole 110 according to the embodiments.

In addition, one end of the light transmission tube 200 may be variously machined according to an area of the radiated UV light, and if necessary, a fine lens may be attached thereto.

As illustrated in FIG. 2, a UV blocking material 321 which blocks the UV light radiated from the optical fiber 200 may be coated on an outer surface of the connection tube 320 of the UV curing resin injection apparatus for treating the aneurysm according to embodiments of the present invention.

When the UV light is transmitted through the outer surface of the connection tube 320, the UV curing resin may be cured in the connection tube 320 and thus close the connection tube 320. Therefore, it is preferable that the UV blocking material 321 is coated on the outer surface of the connection tube 320 to prevent the UV light from being transmitted. Depending on the embodiments, the UV blocking material 321 may be coated on an outer surface of the catheter 100 other than the outer surface of the connection tube 320.

Referring to FIG. 4A, a plurality of curing resin injection orifices 310 may be formed at one end of the nozzle 300 to inject the UV curing resin corresponding to various shapes of the aneurysm. As illustrated in FIGS. 4B and 4C, the curing resin injection orifices 310 may be formed to have different diameters from each other. Therefore, the UV curing resin injection apparatus for treating the aneurysm can be applied to various kinds of operations such as an abdominal aneurysm and a cerebral aneurysm.

In the UV curing resin injection apparatus for treating the aneurysm according to the first embodiment of the present invention, since the light transmission tube 200 and the connection tube 320 are not coupled with each other, but are separated from each other, an injection position of the UV curing resin and a focusing direction and position of the light transmission tube may be controlled during the operation, and freedom of the operation may be enhanced.

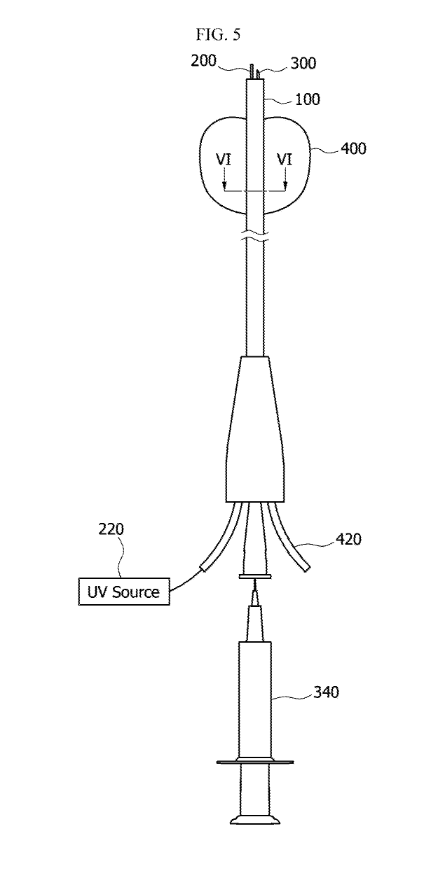

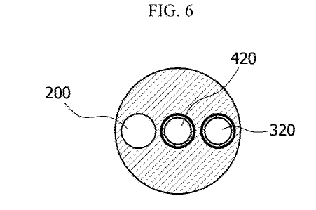

Referring to FIG. 5 which is a front view of a UV curing resin injection apparatus for treating an aneurysm according to a second embodiment of the present invention, and FIG. 6 which is a cross-sectional view taken along line VI-VI of FIG. 1, a balloon 400 may be disposed at one end of the catheter 100.

When a neck of the aneurysm is very wide, the UV curing resin which is injected and cured to close the aneurysm may be discharged to a parent blood vessel through the wide neck.

Therefore, as illustrated in FIG. 5, the balloon 400 may be disposed at the catheter 100, and the UV curing resin may be cured in the aneurysm, and then the balloon 400 may be expanded so that a space between the neck and the balloon 400 becomes narrow. In this case, the UV curing resin cured in the aneurysm may be prevented from being discharged to the parent blood vessel.

As illustrated in FIG. 5, the catheter 100 may be disposed to pass through the balloon 400.

At this time, as illustrated in FIG. 6, a liquid injection tube 420 which is in communication with the balloon 400 to supply a liquid into the balloon 400 and thus to expand the balloon 400 may be disposed between one end and the other end of the catheter 100.

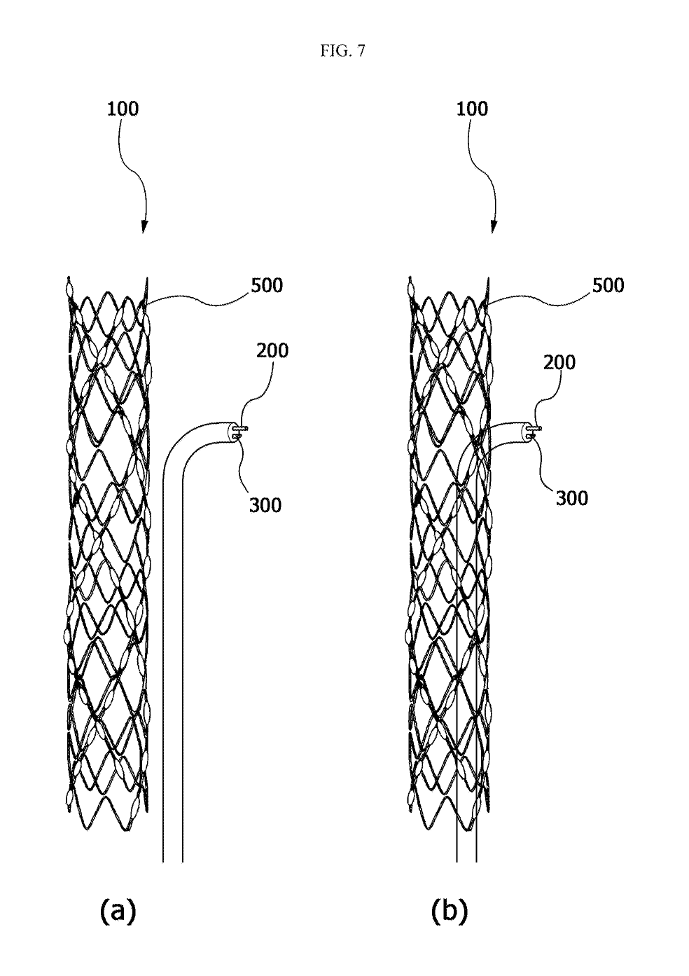

Also, as illustrated in FIG. 7 which is a perspective view of a part of a UV curing resin injection apparatus for treating an aneurysm according to a third embodiment of the present invention, a plurality of wave-shaped modules may be disposed at one side of the catheter 100 to pass through an inner side of a stent 500 having a net shape, or may be disposed at an outside of the stent 500.

In particular, when the neck is not wide and deep, the stent 500 may be inserted into the aneurysm, and the UV curing resin injection apparatus for treating the aneurysm may be inserted through the stent 500, and the UV curing resin may be injected to the neck through a net formed by the stent 500.

In this case, when the neck is wide, as described above, the stent 500 may prevent the UV curing resin cured in the aneurysm from being discharged.

Depending on the embodiments, the stent 500 and the balloon 400 may be used together.

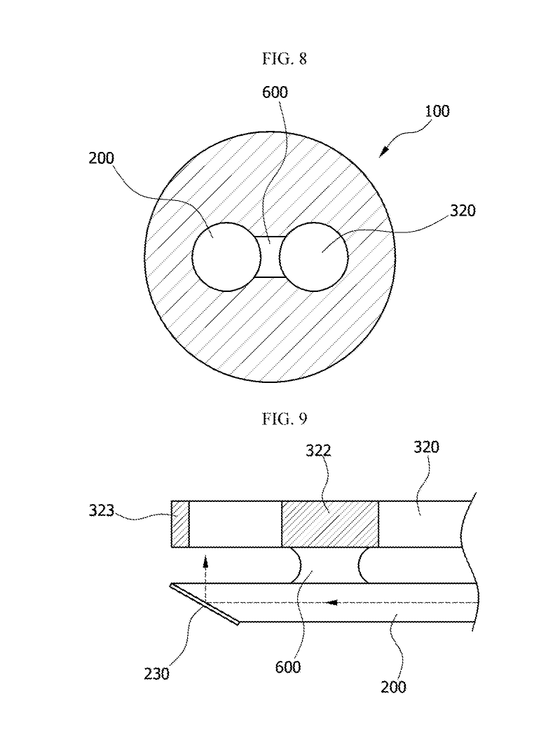

FIG. 8 is a longitudinal cross-sectional view of a catheter 100 of a UV curing resin injection apparatus for treating an aneurysm according to a fourth embodiment of the present invention, and FIG. 9 is a transverse cross-sectional view of the catheter 100 of the UV curing resin injection apparatus for treating the aneurysm according to the fourth embodiment of the present invention.

As illustrated in FIGS. 8 and 9, the UV curing resin injection apparatus for treating the aneurysm according to the fourth embodiment of the present invention includes the catheter 100, the light transmission tube 200, the connection tube 320 and a coupling member 600.

The coupling member 600 is disposed between the light transmission tube 200 and the connection tube 320, and serves to connect the light transmission tube 200 with the connection tube 320.

At this time, the coupling member 600 may connect all of side surfaces of the light transmission tube 200 and the connection tubes 320, or may partly connect the side surfaces of the light transmission tube 200 and the connection tubes 320, as illustrated in

FIG. 9.

Also, an epoxy bond may be used as the coupling member 600.

In particular, in the case of the UV curing resin injection apparatus for treating the aneurysm according to the fourth embodiment of the present invention, since the light transmission tube 200 is fixed to the connection tube 320, unlike the UV curing resin injection apparatus for treating the aneurysm according to the first embodiment of the present invention, the liquid UV curing resin may be prevented from being introduced into a human body, while the light transmission tube is focused on an unnecessary place. Also, since the light transmission tube 200 and the connection tube 320 may be integrally controlled, it is possible to provide convenience to an operator.

Meanwhile, as illustrated in FIG. 9, a reflective surface 230 may be formed at one end of the light transmission tube 200.

The reflective surface 230 may be inclined so that the UV light transmitted through the light transmission tube 200 is reflected in a direction perpendicular to a lengthwise direction of the connection tube 320 and then radiated on the connection tube 320. The reflective surface 230 may be formed by reflection coating.

Meanwhile, in the UV curing resin injection apparatus for treating the aneurysm according to the fourth embodiment of the present invention, the connection tube 320 is movably disposed on an outer circumferential surface of the connection tube 320, and also a first blocking member 322 which blocks the UV light radiated on the connection tube 320 may be further included.

In particular, when the reflective surface 230 is formed at one end of the light transmission tube 200, as described above, only a portion of the connection tube 320, on which the UV light reflected by the reflective surface 230 is radiated, may be opened, and the rest portions may be covered with the first blocking member 322 to block the UV light.

In this case, the UV curing resin may smoothly flow through the portions of the connection tube 320 covered by the first blocking member 322 to block the UV light, and then may be cured at the portion thereof in which the first blocking member 322 is not disposed and thus the UV light is not blocked.

Therefore, as illustrated in FIG. 9, when the UV light is radiated on only the portion just before the UV curing resin is injected using the first blocking member 322, the UV curing resin may be somewhat cured in the connection tube 320 before being injected, and thus the UV curing resin injected into the aneurysm may be completely cured in a shorter time.

However, the portion in which the UV light is blocked by the first blocking member 322 and the portions in which the UV light is not blocked should be appropriately set so that the UV curing resin is prevented from being completely cured in the connection tube 320 and blocking the connection tube 320.

Therefore, to easily set the portion in which the UV light is blocked by the first blocking member 322, the first blocking member 322 may be movably disposed on the outer circumferential surface of the connection tube 320. In particular, when the portion in which the UV light is blocked is set, a kind of the UV curing resin, and a kind and intensity of the light transmission tube 200 should be considered comprehensively.

However, even when the portion in which the UV light is blocked by the first blocking member 322 is appropriately set, there is a risk that the injection orifice located at one end of the connection tube 320 is blocked by curing of the UV curing resin. In particular, when the injection orifice has a small diameter, more serious problems may occur.

Thus, in embodiments, a second blocking member 323 which blocks the UV light radiated on the connection tube 320 may be further included at an outer circumferential surface of one end of the connection tube 320.

Meanwhile, when the UV curing resin is injected and cured in the aneurysm and then the UV curing resin injection apparatus for treating the aneurysm is removed, the UV curing resin injection apparatus may be adhered with the UV curing resin, and may be difficult to be removed.

Thus, in embodiments, a plurality of micro-protrusions may be formed at a cross section and an outer circumferential surface of one end of the connection tube 320.

Further, a chemical finishing method may be applied to the cross section and the outer circumferential surface of one end thereof so that the one end is not adhered with the

UV curing material.

In the chemical finishing method, a material of the connection tube 320 and a material of the UV curing material should be considered. In addition, to perform a function of the second blocking member 322 together, the micro-protrusions may be formed on the cross section and the outer circumferential surface of one end of the connection tube 320, or the chemical finishing method may be applied thereto.

In FIGS. 1 to 9, only the catheter 100 of which one end and the other end are formed in a straight line, or one end is bent is illustrated. However, the shape of the catheter 100 is just a preferable example for the purpose of illustrations only, and may have various shapes such as a “J” shape and an “S” shape according to the shape of the aneurysm.

Although embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A UV curing resin injection apparatus for treating aneurysm, comprising:
a catheter;
a light transmission tube extending inside the catheter and configured to transmit light beams therethrough; and
a resin transfer tube extending inside the catheter and configured to transfer uncured resin therethrough,
wherein the resin transfer tube and the light transmission tube are independently formed and spaced from each other inside the catheter

2. The apparatus of claim 1, wherein the light transmission tube comprises an optical fiber, wherein one end of the light transmission tube protrudes out of the catheter, wherein the other end of the light transmission tube is operably linked to a UV source for receiving UV light beams therefrom.

3. The apparatus of claim 2, wherein the catheter comprises a channel formed through the catheter, wherein the light transmission tube extends inside the channel.

4. The apparatus of claim 3, wherein the light transmission tube is movable relative to the channel of the catheter.

5. The apparatus of claim 1, further comprises a nozzle provided at one end of the resin transfer tube, wherein the other end of the resin transfer tube is in communication with a uncured resin supply.

6. The apparatus of claim 5, wherein the uncured resin supply comprises a pump.

7. The apparatus of claim 5, wherein the nozzle comprises a plurality of injection orifices.

8. The apparatus of claim 7, wherein at least part of the plurality of injection orifices have different diameters from each other.

9. The apparatus of claim 1, wherein the resin transfer tube is coated with a UV blocking material.

10. The apparatus of claim 1, further comprising:
a balloon connected to the catheter and
a balloon-inflating fluid tube extending inside the catheter and in communication with the balloon.

11. The apparatus of claim 1, wherein the light transmission tube comprises a distal end portion extending outside the catheter, the resin transfer tube comprises a distal end portion extending outside the catheter.

12. The apparatus of claim 11, wherein the distal end portions of the light transmission tube and the resin transfer tube are coupled by a coupling member disposed therebetween.

13. The apparatus of claim 11, further comprising a reflective surface provided in the distal end portion of the light transmission tube, wherein the reflective surface is inclined relative to a direction of elongation of the light transmission tube such that the light beams reaching the reflective surface are directed toward the distal end portion of the resin transfer tube.

14. The apparatus of claim 13, wherein the reflective surface comprises a reflective coating.

15. The apparatus of claim 13, wherein the distal end portion of the resin transfer tube is configured to receive light beams from the light transmission tube such that the uncured resin in the distal end portion of the resin transfer tube is exposed to the light beams and begin curing prior to being discharged from the distal end portion of the resin transfer tube.

16. The apparatus of claim 15, further comprising a light blocking member movably placed over an outer circumference of the distal end portion of the resin transfer tube, wherein the light blocking member is configured to block light beams from the distal end portion of the light transmission tube, wherein the light blocking member is configured to slide over the outer circumference of the distal end portion of the resin transfer tube such that exposure to the light beams of the uncured resin inside the distal portion of the resin transfer tube can be adjusted.

17. The apparatus of claim 11, further comprising a light blocking member disposed over an outer circumferential area of the distal end portion of the resin transfer tube where a nozzle is provided.

18. The apparatus of claim 11, further comprising a plurality of micro-protrusions formed on an outer circumferential surface of the distal end portion of the resin transfer tube.

19. A system comprising:
the apparatus of claim 1;
a stent to use with the apparatus, the stent comprising a plurality of wave-shaped modules, wherein the apparatus is to be placed at an inner side or an outer side of the stent.
